(19) United States
(12) Patent Application Publication
(10) Pub. No.: US 2024/0191268 A1
(43) Pub. Date: Jun. 13, 2024

(54) AN ENZYMATIC PRE-TREATMENT METHOD FOR EFFICIENT SACCHARIFICATION OF LIGNOCELLULOSIC MATERIALS

(71) Applicant: PRAJ INDUSTRIES LIMITED, Pune (IN)

(72) Inventors: Phaneeswara RAO KOMMOJU, Pune (IN); Apoorva DESHMUKH-KAMBALE, Pune (IN); Parnal SUDHIR SATTIKAR, Pune (IN); Dipesh DATTU JADHAV, Pune (IN); Geetanjali SWAROOP WAKADE, Pune (IN); Nikhil DEORAO AMBATKAR, Pune (IN); Pramod SHANKAR KUMBHAR, Pune (IN)

(21) Appl. No.: 18/286,938

(22) PCT Filed: May 6, 2022
(86) PCT No.: PCT/IN2022/050439

§ 371 Date: Oct. 13, 2023

(30) Foreign Application Priority Data
May 7, 2021 (IN) 202121020751

(51) Int. Cl. C12P 19/04; C12N 9/04; C12N 9/42; C12P 19/02; C12P 19/14; C12R 1/82

(52) U.S. Cl. CPC C12P 19/04; C12N 9/0006; C12N 9/2437; C12P 19/02; C12P 19/14; C12P 2201/00; C12P 2203/00; C12R 2001/82; C12Y 111/01013; C12Y 111/01014; C12Y 111/01016; C12Y 302/01008

(57) ABSTRACT

The presently disclosed invention relates to an enzymatic pre-treatment method for efficient saccharification of lignocellulosic materials. The invention involves a process for releasing carbohydrates/sugars from processing the agricultural feedstock and pre-treating the processed feedstock with lignin modifying enzymes (LME) to produce the reaction mixture which is further used for releasing free carbohydrates/sugars. More particularly, it relates to the production of lignin modifying enzymes and efficient delignification of lignocellulosic materials by using lignin modifying enzymes to prepare ethanol or other bio-chemicals.

### TECHNICAL FIELD

The invention relates to a method for increasing enzymatic hydrolysis of agricultural feedstock. More particularly, it relates to use of lignin modifying enzymes for enhancing efficiency of saccharification to release carbohydrates/sugars.

### BACKGROUND

Increasing demand world over for fuel energy and its gradual depletion due to limited availability of fossil fuels, particularly crude oil, necessitates the development of renewable and alternate sources for obtaining fuel, such as biofuels, that may be applied for running automobiles and machinery. In India, bioethanol is being primarily produced by microbial fermentation of sugars. Presently, the largest source of sugars for bioethanol production is plant-derived lignocellulosic material (LCM) and waste biomass from agricultural feedstock.

While the availability of raw biomass varies widely on the source and the time of year, woody biomass is more likely to be harvested year-round and agricultural residues and grasses harvested on a seasonal basis. However, for the purpose of the present invention, agricultural feedstock and plant-derived biomass have been used synonymously to encompass any renewable source that comprises lignocellulosic material.

LCM is a complex structure of cellulose, hemicellulose and lignin forming a composite which depending on its source is differentially resistant to hydrolysis compared with other carbohydrate-based materials like starch. Several methods are known in the art for the pre-treatment of LCM such as chemical and thermal hydrolysis involving use of hot water, acid, alkali and other chemicals; performed to achieve effective degradation of LCM to release fermentable sugars—a process referred to as saccharification that provides free carbohydrates/sugars-like xylose and glucose. These sugars on fermentation by yeasts produce ethanol that is used in various applications including as a fuel additive.

While pre-treating agricultural feedstock with dilute acid at elevated temperature and pressure is a widely employed due to its low cost, ease of unit operation and good separation of the C5 sugars, its major shortcoming stems from a low C6 enzymatic hydrolysis efficiency (maximum 50%) plausibly due to the presence of soluble lignin and inhibitors generated during the pre-treatment. Soluble lignin irreversibly binds to cellulolytic enzymes such as cellulase, deactivating it and consequently lowering the efficiency of such hydrolysis. Hence, an effective and improved method to eliminate or reduce enzyme-lignin interactions and thereby increase the efficiency of cellulolytic enzymes is highly desirable. Use of biocatalysts to modify lignin and thus enhance its removal from the hydrolysate is an attractive option for reducing enzyme-lignin interactions.

Lignin modification using lignin-modifying enzymes (LME) or lignocellulolytic enzymes from the fungal secretome is widely known. In nature, white-rot fungi secrete LMEs and are the most effective degraders of lignin and differ from the other wood-decaying basidiomycetes, named soft and brown rot fungi, for their ability to degrade lignin along with cellulose and hemicellulose. LMEs include peroxidases such as lignin peroxidase (Lip), manganese peroxidase (MnP), versatile peroxidases (VP), laccases etc.

Laccases are blue multicopper oxidases that catalyse the oxidation of substituted phenols concurrently with the reduction of molecular oxygen to water. MnP catalyse H₂O₂-dependent oxidation of Mn²⁺ to highly reactive Mn³⁺ that once chelated with carboxylic acids, diffuses into the phenolic lignin structure leading to the formation of free radicals that decompose spontaneously. Further, MnP also oxidize non-phenolic substrates only in the presence of a second mediator, whereas LiP are known to oxidize both phenolic and non-phenolic lignin units. VP display a unique ability to oxidize compounds including high-redox-potential aromatic compounds, besides those oxidized by MnP and LiP.

In the present invention, the LME produced by white rot fungi were used to modify and remove lignin from agricultural feedstock resulting in a pre-treated biomass that was easily accessible to saccharifying cellulolytic enzymes thus leading to increased concentration of free carbohydrates/sugars.

### SUMMARY

The presently disclosed subject matter provides a process for releasing carbohydrates/sugars from agricultural feedstock, the process comprising: processing the agricultural feedstock; pre-treating the processed feedstock with lignin modifying enzymes (LME) to produce a reaction mixture; separating the reaction mixture into solids and liquid comprising LME; washing the solids with at least one of water, alkaline peroxide or ionic liquid to obtain washed solids free from LME; and saccharifying the washed solids by hydrolysis with one or more cellulolytic enzymes to release free carbohydrates/sugars at an increased efficiency.

In an exemplary embodiment, the instant invention discloses a process for pre-treatment of agricultural feedstock or biomass wherein the steps include the selection of microorganisms, inoculum preparation, optimization of the media composition pre-treatment and enzymatic saccharification, conversion efficiency, of cellulose to glucose with or without water wash, conversion efficiency of cellulose to glucose with and without peroxide treatment, determination of LME remaining after pre-treatment, LME recycle, efficiency of recycled LME.

This summary is not intended to identify all the essential features of the claimed subject matter, nor is it intended to use in determining or limiting the scope of the claimed subject matter.

### BRIEF DESCRIPTION OF DRAWINGS

The detailed description of the drawings is outlined with reference to the accompanying figures. In the figures, the left-most digit (s) of a reference number identifies the Figure in which the reference number first appears. The same numbers are used throughout the drawings to refer like features and components.

FIG. 1 demonstrates the flow chart for an enzymatic pre-treatment method for efficient saccharification of lignocellulosic materials

FIG. 2 demonstrates qualitative plate assay for the selection of microorganisms

FIG. 3 demonstrates SEM photos of rice straw treated with water (control) and Trametes versicolor laccase

### DETAILED DESCRIPTION

Before the present process or steps are described, it is to be understood that this disclosure is not limited to the particular process or organism, as there can be multiple possible embodiments which are not expressly illustrated in the present disclosure but may still be practicable within the scope of the present disclosure.

### Definitions

For better representation and understanding of the present invention, several terms have been defined.

Reference throughout the specification to “related embodiments,” “one embodiment,” or “various embodiments” means that a particular feature, structure, or characteristic described in connection with the embodiment is included in at least one embodiment. Thus, appearances of the phrases “in preferred embodiment,” “in one embodiment,” or “in an embodiment” in places throughout the specification are not necessarily all referring to the same embodiment. Furthermore, the particular features, structures or characteristics may be combined in any suitable manner in one or more embodiments.

The term “bioethanol” is used interchangeably herein with “ethanol” and refers to ethanol generated from the “conversion” of plant matter. As used herein, the term “conversion” refers to the process of producing ethanol from a biomass.

The term “biomass”, as used herein, refers to any plant matter including a part or a piece of any of a variety of plant species for use in the production of ethanol. In certain embodiments, the biomass comprises plant material from more than one species of plants.

The term “delignification” refers to the removal of some or all of the lignin present in a lignin-containing sample. Delignification may be performed via chemical, mechanical, or enzymatic processes or combinations thereof.

The terms “fermentable sugar” or “free sugar” or “fermentable carbohydrate” or “free carbohydrate” can be used interchangeably and refer to oligosaccharides, monosaccharides and mixtures thereof that can be used as a carbon source in a fermentation process.

“Hydration” and “hydrate”, as used herein, refer to an increase in and/or increasing the moisture content of a biomass such as by, e.g., soaking or steaming or by hot water treatment.

The terms “hydrolyse” or “hydrolysis” and variations thereof refer to the process of converting polysaccharides (e.g., cellulose) to fermentable sugars, e.g., through the hydrolysis of glycosidic bonds. This process is also referred to as saccharification. Hydrolysis may be affected with enzymes or chemicals, either by directly adding to the biomass (as a solid or liquid enzyme additive) or may be produced in-situ by microbes (such as yeasts, fungi, bacteria, etc.). Hydrolysis products include fermentable sugars such as glucose and other small (low molecular weight) oligosaccharides such as monosaccharides, disaccharides, and trisaccharides.

“Lignin” is a polyphenolic material comprised of phenyl propane units linked by ether and carbon-carbon bonds that may be highly branched and crosslinked.

“Cellulose” is a polysaccharide of β-glucose comprising β-(1-4) glycosidic bonds and “cellulolytic” refers to anything having the capacity to hydrolyse cellulose. “Hemicellulose” refers to polysaccharides comprising mainly sugars or combinations of sugars other than glucose (e.g., xylose) and may be highly branched, chemically bonded to lignin, and randomly acetylated that may reduce hydrolysis or enzymatic hydrolysis of the glycosidic bonds in hemicellulose.

“Enzyme” refers to protein that catalyses conversion of one molecule into another and herein includes any enzyme that catalyses the transformation of a biomass-derived molecule to another biomass-derived molecule and encompass those that degrade, hydrolyse or otherwise transform saccharide, cellulose, or lignocelluloses to provide fermentable free sugars/carbohydrates and/or alcohols.

The terms “lignocellulolytic enzymes”, “lignin modifying enzymes” or “LME” or “cellulolytic enzyme” or “cellulolytic enzyme” refers to enzymes that are involved in the disruption and/or degradation of lignocellulose. The disruption of lignocellulose leads to the formation of substances including monosaccharides, disaccharides, polysaccharides and phenols. Lignocellulosic enzymes include, but are not limited to, cellulases, hemicellulases and ligninases. Thus, lignocellulosic enzymes include saccharification enzymes, i.e., enzymes which hydrolyse (i.e., depolymerize) polysaccharides.

The term “cellulase’ when used generally refers to enzymes involved in cellulose degradation. Similarly, hemicellulases are involved in hemicellulose degradation. “Hemicellulases” include xylanases, arabinofuranosidases, acetyl xylanesterases, glucuronidases, mannanases, galactanases, and arabinases. “Ligninases” are enzymes that are involved in the degradation of lignin. A variety of fungi and bacteria produce ligninases. Lignin-degrading enzymes include, but are not limited to, lignin peroxidases, manganese-dependent peroxidases, hybrid peroxidases (which exhibit combined proper ties of lignin peroxidases and manganese-dependent peroxidases), and laccases.

The term “slurry”, as used herein, refers to the liquid and solid components of the biomass generated from pre-treatment of the biomass.

“Suitable conditions” for alcohol fermentation refers to conditions that support the production of ethanol or another alcohol by a biocatalyst.

Such conditions include but are not limited to pH, nutrients, temperature, atmosphere, and other factors.

The technical solutions offered by the present disclosure are clearly and completely described below. Examples in which specific compounds or conditions may not have been specified have been conducted under conventional conditions or in a manner recommended by the manufacturer.

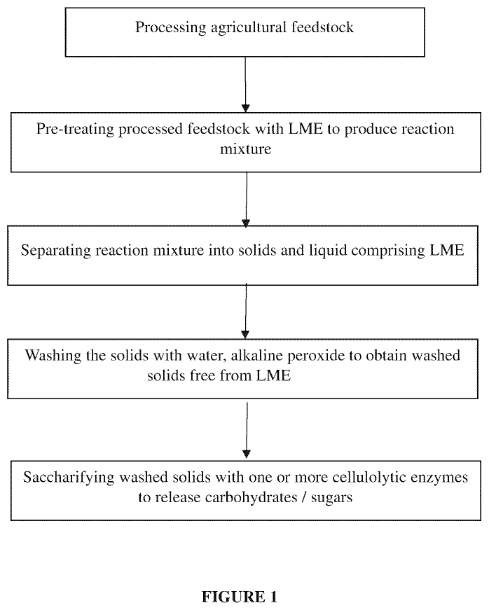

In a preferred embodiment, the presently disclosed subject matter provides a process for releasing carbohydrates/sugars from agricultural feedstock, the process comprising: processing the agricultural feedstock; pre-treating the processed feedstock with lignin modifying enzymes (LME) to produce a reaction mixture; separating the reaction mixture into solids and liquid comprising LME; washing the solids with at least one of water, alkaline peroxide or ionic liquid to obtain washed solids free from LME; and saccharifying the washed solids by hydrolysis with one or more cellulolytic enzymes to release free carbohydrates/sugars at an increased efficiency (see FIG. 1).

In one embodiment of the present invention, the process includes several steps. Each step has one or more elements for performing specific function as required for production of LME for modifying the lignin before enzymatic hydrolysis. A person skilled in the art may appreciate different variations and/or combinations of these elements that may be used to perform the objects of the invention disclosed herein.

In some embodiments, the agricultural feedstock or biomass is selected from but not limited to the group of herbaceous material, agricultural residues, forestry residues, wastepaper, pulp and paper mill residues, or a combination thereof. In some embodiments, the agricultural feedstock is selected from the unlimiting group comprising corn stover, rice straw, wheat straw, sugarcane, beet bagasse, miscanthus, sorghum residue, switch grass, bamboo, water hyacinth, hardwood, hardwood chips, softwood chips, hardwood pulp, and softwood pulp, corncob, corn stover, sugarcane/beet bagasse or any similar lignocellulosic materials using lignin modifying enzymes (LME). In a related embodiment, the agricultural feedstock comprises one or more of rice straw, wheat straw, corncob, corn stover, sugarcane/beet bagasse or combinations thereof.

In one embodiment of the invention, white rot fungi are used for the production of lignin modifying enzymes selected, but are not limited to Trametes versicolor, Trametes trogii, Trametes villosa, Pleurotus ostreatus, Pleurotus pulmonarius Pleurotus calyptratus, Trichoderma asperllum, Phanerochaete chrysosporium, and Peniillium chrysogenum. In a preferred embodiment, the lignin modifying enzymes (LME) are produced by fermentation of white rot fungi strains selected from Trametes versicolor, Pleurotus ostreatus Phanerochaete chrysosporium, and Penicillium chrysogenum.

In accordance with an exemplary embodiment of the present disclosure, white rot fungi Trametes versicolor and Pleurotus ostreatus are selected by qualitative (plate assay) and quantitative (submerged fermentation) assays. In another embodiment, the said cultures are used for the production of LME and the detection of LME's was carried out by indicator plate method.

In an exemplary embodiment, fungal strains grown for 5 days at 25° C. on agar plugs on yeast extract-malt extract (YM) plates comprising indicators and dye, and enzyme substrates allowed detection of LME producers. The indicators—0.1% (w/v) ABTS and 0.01% (v/v) guaiacol—are used to detect the presence of laccase and manganese peroxidase respectively while 0.01% (w/v) Azure B dye detected the presence of lignin peroxidase. Formation of greenish colour zone due the oxidation of ABTS shows presence of laccase while a zone of reddish-brown colour indicates presence of manganese peroxidase due to the oxidation of guaiacol. Plates with azure B dye show a decolorization zone since lignin peroxidase is known to oxidise azure B dye and form colourless product (see FIG. 1)

In one embodiment, the present invention discloses the preparation of lignin modifying enzymes (LME) comprising the steps of inoculum preparation and media optimization. Solid State Fermentation (SSF) is an important mode of fermentation where microorganisms are grown on solid substrates in the absence or near-absence of free water and secrete the desired product efficiently. In one embodiment, the inoculum comprises solid medium like rice straw or rice or other suitable solids or agriculture by-products. The said inoculum is used for LME production.

LME are produced using surface, static or submerged fermentation. In the present disclosure, LME are produced using white rot fungi in an optimised media broth employing submerged fermentation under suitable standardised conditions like temperature. pH, oxygen and carbon dioxide levels. Desired bioactive compounds are secreted into the fermentation broth. The said fungi showed poor LME expression in a liquid medium containing sucrose (1%), yeast extract (0.4%) and malt extract (1%).

In a preferred embodiment, the submerged fermentation media comprises molasses, yeast extract, corn steep liquor, sodium lignosulphonate and FeSO₄.

Amongst others, media optimization is a critically investigated factor—involving a process wherein the media components or fermentation conditions are varied by varying concentrations or modifying conditions to obtain better growth of the organisms along with high productivity of desired bioactive. This has a significant impact on growth and production kinetics of a bioprocess.

In an embodiment, the media was further optimised by optimizing media and fermentation parameters using full factorial design to achieve maximum product concentration. Further, as media components may aid productivity by increasing flux through a metabolic node, and impacting performance of a strain, a focus on media optimization yields significant performance gain. In a preferred embodiment of the present disclosure, the submerged fermentation is carried out from 7 to 25 days at a pH ranging from 5.5 to 6.5 and at a temperature ranging from 18 to 37° C. by inoculating the fermentation media with 5 to 10% active mycelia to provide LME.

Presence of inducers may aid production of the desired product in acceptable yields, titres, and volumetric productivity. In a related embodiment, the fermentation media optionally further comprises one or more inducers added at suitable times during the fermentation; the inducers selected from copper sulphate, wheat straw extract, rice straw extract, lignosulphonate, and 2,5-xylidine.

Fermentation is followed by the separation of solids and liquid using methods selected from but not limited to evaporation, filtration, sedimentation, crystallization for the recovery of valuable solid component or liquid recovery or where recovery of both solid and liquid is performed as necessitated by the nature of the desired product/phase. In an embodiment, fungal mycelia and other solids are separated using solid liquid separation to provide clear broth containing LME that is used for pre-treatment of biomass.

In one embodiment, the present invention discloses a process for releasing carbohydrates/sugars from agricultural feedstock, the process comprising pre-treating the processed feedstock with LME to produce the reaction mixture.

Pre-treatment is critical for optimal cellulose hydrolysis, to make cellulose more available to cellulolytic enzymes that hydrolyse the biomass polymers into free fermentable sugars or carbohydrates. Pre-treatment of lignocellulosic material is carried out to overcome recalcitrance through the combination of chemical and structural changes to the lignin and carbohydrates.

Prior to pre-treatment, the agriculture feedstock is processed using one or more methods selected from but not limited to autohydrolysis, grinding, chopping, treating with hot water or dilute acid. In a preferred embodiment, processing of the agricultural feedstock involves one or more steps of treating with hot water or mild acid, drying and mechanical shredding to provide solid particles. In a further embodiment, processing comprises mechanical reduction of size of solid particles; the processed feedstock comprising solid particles with sizes in a range of 20 to 60 mm, and preferably in the range of 30 to 50 mm, more preferably, the solid particles are reduced to sizes <40 mm. Alternatively, the feedstock is hydrated.

In a further embodiment, the processed feedstock is pre-treated with LME to produce a reaction mixture. Pre-treating of the processed feedstock includes mixing the processed feedstock with LME for 10 to 48 hours at a temperature ranging from 40° C. to 50° C. to produce the reaction mixture.

In another embodiment, the reaction mixture comprising solids obtained from the pre-treatment of processed feedstock by LME is washed with at least one of water, alkaline peroxide or ionic liquid. The said solids are washed to remove the remaining LMEs, to obtain the washed solids which are free from LME. In a preferred embodiment, washing the solids comprises one or more washes to provide the washed solids free from LME.

In a preferred embodiment, separating the reaction mixture includes filtration, centrifugation, decantation methods to provide solids and liquid comprising LME. In a related embodiment, the liquid comprising LME is recycled and reused for the next batch.

In some embodiments, processing of the biomass comprises hydration for biomass preparation, making it conducive to enzymatic saccharification reaction. Further treatment may involve chemical treatment with an alkali selected from sodium hydroxide, potassium hydroxide, calcium hydroxide, sodium carbonate and sodium hydrogen carbonate, or a mixture thereof, or a mixture of sodium sulfite.

In a related embodiment, the said solids are washed with water and/or followed by treatment with alkaline peroxide before subjecting it to enzymatic hydrolysis or saccharification at desired temperature and pH.

In a preferred embodiment, washing of the solids with alkaline peroxide comprises 0.2% to 1% w/w NaOH and 0.1% to 2% w/w H₂O₂ based on cellulose content at a temperature ranging from 60° C. to 80° C. for 60 to 90 minutes to obtain washed solids.

In a preferred embodiment, saccharifying the washed solids by hydrolysis with one or more cellulolytic enzymes to release carbohydrates/sugars at an increased efficiency involves saccharification under suitable conditions like optimal biomass composition, enzyme composition, pH and temperature. The enzymes may be added directly or produced in-situ to release fermentable free sugars/carbohydrates like glucose, xylose and other low molecular weight oligosaccharides like monosaccharides, disaccharides, and trisaccharides.

In some embodiments, one or more cellulolytic enzymes may comprise a commercially available enzyme cocktail to produce the monosaccharides that are used in the biosynthesis of fermentation end-products. In a preferred embodiment, saccharifying the washed solids involves hydrolysis with one or more cellulolytic enzymes selected from cellulases, hemicellulases or xylanases.

In an embodiment, saccharifying the washed solids by hydrolysis with one or more cellulolytic enzymes is carried out at a temperature ranging from 30° C. to 90° C., preferably from 35° C. to 85° C. and more preferably from 40 to 80° C. The pH isoptimized based on the type of enzymes being used. In some embodiments, the pH may be in the acidic range between 3 to 6, preferably between pH 4 and 5. In some embodiments, the pH is adjusted to about 4.8 using acids, bases or buffers so long as they do not adversely affect enzyme function. In a preferred embodiment, cellulolytic hydrolysis involves mixing the washed solids with one or more of cellulase, hemicellulase or xylanase to obtain a slurry having 5% to 15% w/w solids at pH 4.5 to 5.5 and temperature of 40° C. to 80° C. for 24 to 120 hours to obtain a final stream comprising free carbohydrates/sugars. Commercial cellulolytic enzymes are required in less amount due to delignification.

An increase in the rate of hydrolysis or in cellulose accessibility or alteration of the structure of cellulose to a less stable form is desirable. Accessory proteins are known to facilitate cellulose fibre disaggregation for subsequent cellulase action. Non-hydrolytic “loosening” or disruption of a cellulosic substrate is being increasingly recognized as one of the main stages of enzymatic deconstruction of cellulosic biomass along with identification of several non-hydrolytic disruptive proteins. In one embodiment, accessory proteins include but are not limited to swollenin, loosenin, expansins, expansin-like proteins, lytic polysaccharide monooxygenases (LPMOS), fibril forming protein, lytic polysaccharide monooxygenases (AA9) etc.

In a preferred embodiment, saccharifying the washed solids involves hydrolysis, involving addition of one or more accessory proteins selected from expansins, swollenins and lytic polysaccharide monosaccharides (LMPO) such as AA9. In some embodiments, the hydrolysis or saccharification efficiency is 30% or greater; between 35% and 85%. In a preferred embodiment, the hydrolysis efficiency is in the range of 40% to 80%.

“Fermentation” or “fermenting” refers to the process of transforming an organic molecule into another molecule using a microorganism. Fermentation may be aerobic such as alcohol fermentation or of anaerobic nature. Product of an alcohol fermentation is determined by the biocatalyst used and/or the substrate of fermentation. In certain embodiments, fermenting comprises contacting a mixture including biomass-derived or free carbohydrates/sugars with an alcohol-producing biocatalyst, such as a yeast or other alcohol-producing microbes.

In another embodiment, enzymes or biocatalysts are added to the fermentation or are produced by microorganisms present in the fermentation. In various embodiments, the microorganism is yeast. In some embodiments, the obtained alcohol mixture is preferably 2 to 5% alcohol by volume.

In one embodiment, after the saccharification, yeast is added to convert the free sugars to ethanol under continuous agitation for 15 to 90 h; preferably 20 to 80 h and more preferably 24 to 72 h. In a related embodiment, the fermentation is maintained from about 20° C. to 40° C. and more preferably at 30° C.

In a preferred embodiment, the released free carbohydrates/sugars are further fermented using yeast for 24 to 72 h at 30° C. with continuous agitation to obtain ethanol. Alternatively, other biochemicals may also be produced including but not limited to 3-hydroxy propionic acid, glycerol, lactic acid, malonic acid, propionic acid, serine, 3-hydroxy butyrolactone, acetoin, aspartic acid, fumaric acid, malic, succinic acid, threonine, arabitol, furfural, glutamic, itaconic acid, levulinic acid, xylitol, 2,5 furan dicarboxylic add, aconitic acid, citric add, glucaric etc. In a related preferred embodiment, the released carbohydrates/sugars are used for producing other biochemicals including furfural, HMF, lactic acid or 2,3 BDO.

In a related embodiment, the washings resulting from water wash having the residual enzyme collected after biomass treatment is recycled to the clear LME stream and reused in the next cycle of biomass pre-treatment.

In one embodiment, the washings comprise residual laccase in the range of 20 to 80%. In another embodiment, the residual laccase may be in the range of 30 to 70%. In a preferred embodiment, the separated liquid comprising LME comprises about 40 to 60% laccase that is recycled back for next cycle of biomass pre-treatment. It is recycled with fresh load of LME as per dosage requirement. Structural changes are observed on the surface of the biomass or solids when treated with the laccase (FIG. 3).

Accordingly, the subject matter of the present invention also provides methods for producing alcohol from agricultural feedstock wherein the method comprises enzyme hydrolysis of a high consistency biomass mixture, as well as compositions comprising alcohol produced thereby.

Certain objectives of the presently disclosed subject matter has been stated hereinabove, which are addressed in whole or in part by the presently disclosed subject matter, other objectives and advantages will be apparent to skilled persons in the art from the accompanying non-limiting examples.

### EXAMPLES

The following examples have been included to provide illustrations of the presently disclosed subject matter. In light of the present disclosure and the general level of skill in the art, those of skill will appreciate that the following examples are intended to be exemplary and include numerous changes, modifications and alterations that may be employed without departing from the spirit and scope of the presently disclosed subject matter.

### Example 1

Source, maintenance and of cultures: Five fungal strains were tested and include Penicillium sp. and Phanerochaete sp. obtained from Praj matrix culture collection centre (PMCC), Pleuorotus ostreatus, Trametes versicolor sp. obtained from Aloha medicinals, USA and Trichoderma sp. obtained from Microbial Type Culture Collection centre (MTCC) Chandigarh, India Spores of Penicillium sp., Phanerochaete sp., and Trichoderma sp. were stored at −80° C. as glycerol stocks and when required were grown in potato dextrose broth (PDB). Pleurotus ostretus and Trametes versicolor were stored in the form of mycelia supplemented with peptone at final concentration of 10% and stored at −80° C. until further use. For use, all the strains were maintained on potato dextrose agar (PDA) plates and were revived on PDA plates in 7 days at 25° C. prior to use.

### Example 2

Selection of culture: The fungi were selected based on their ability to produce lignin modifying enzymes (LME) as assessed using different indicators. Filter sterilized indicators and dyes were added to autoclaved agar (2% w/v) and poured into glass petri dishes (90 mm). All fungal strains were grown on 8 mm diameter agar plugs on yeast extract-malt extract (YM) media plates. Inoculated plugs were incubated at 25° C. for 5 days in a desiccator and later transferred on to the plates containing respective enzyme substrates or indicators or dyes and further incubated in a desiccator for 5 days at 25° C. Detection of LME produced by the fungi was carried out using 0.1% (w/v) ABTS and 0.01% (v/v) guaiacol to detect the presence of laccase and manganese peroxidase respectively. 0.01% (w/v) Azure B dye was used to detect the presence of lignin peroxidase.

Formation of greenish colour zone due the oxidation of ABTS indicated the presence of laccase; while presence of manganese peroxidase was detected by the formation of a reddish-brown coloured zone due to the oxidation of guaiacol. Plates with azure B dye showed a decolorization zone as lignin peroxidase oxidised azure B dye to form a colourless product.

TABLE 1
List of microorganisms used for the qualitative plate assay

Zone diameter (cm)

| Strains of | Laccase | MnP | LiP |
|---|---|---|---|
| microorganism | (ABTS) | (Guaiacol) | (Azure B) |
| Trametes versicolor | 5.1 ± 0.1 | 5.8 ± 0.1 | 6.0 ± 0.1 |
| Pleurotus ostreatus | 4.7 ± 0.3 | 2.5 ± 0.1 | 2.9 ± 0.1 |
| Trichoderma asperllum | 7.7 ± 0.3 | 0.8 ± 0.1 | 2.6 ± 0.2 |
| Phanerochaete | 4.6 ± 0.1 | — | 4.5 ± 0.1 |
| chrysosporium | | | |
| Penicillium chrysogenum | — | — | 2.2 ± 0.1 |

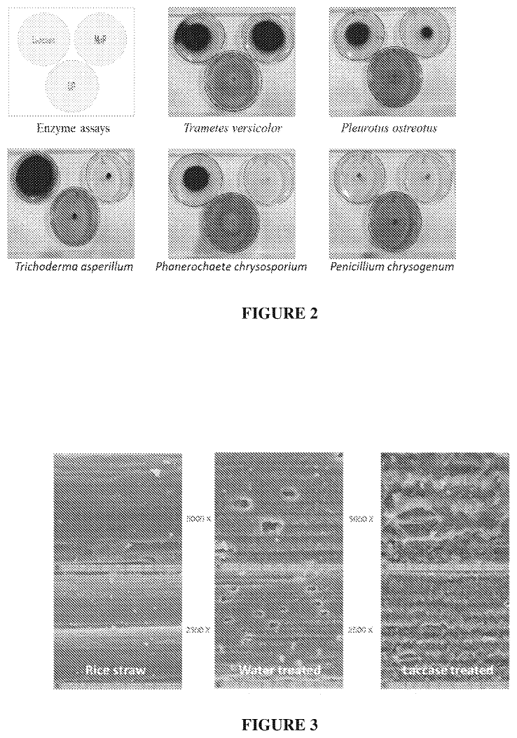

From the obtained results, (see Table 1 and FIG. 2), Trametes versicolor and Pleurotus ostreatus were selected for the production of LMEs.

### Example 3

Inoculum preparation: Frozen mycelia of Pleurotus ostreatus were grown in 150 mL potato dextrose broth (PDB) at 25° C. in a shaker incubator. 4-day old culture was further grown in 350 mL PDB for 4 days for obtaining the 2^{nd} stage seed. Main fermentation (350 mL) was inoculated with 10% of 2^{nd} stage seed and incubated for 20-30 days. Laccase activity was monitored at 7-days intervals. LME activity was measured in terms of laccase units (U/L) and an activity of 2041 U/L was observed for a 20-day culture comprising SYM medium.

Addition of various inducers like CuSO₄, sodium lignosulphonate, and wheat straw extract as depicted in Table 2 resulted in enhanced laccase secretion and activity.

### Example 4

Media Optimization: Media was optimized after full factorial design (FFD) using one or more of molasses, corn steep liquor, and tween 80 and addition of inducers. Adding tween 80 (w/v) (0.5%) and CuSO₄ (0.2 mM) demonstrated laccase activity of 23889 U/L in a 20-day fermentation. Further, SLS media comprising total sugars (56.2 g/L) from cane molasses, yeast extract (1 g/L), corn steep liquor (27 g/L), FeSO₄ (0.56 g/L) and adding CuSO₄ (1.2 mM), sodium lignosulphonate (10 g/L) as inducers yielded highest laccase activity of 1,45,665 U/L in 25 days. Further optimization with SWX media comprising total sugars (56.2 g/L from cane molasses), yeast extract (1 g/L), corn steep liquor (27 g/L), FeSO₄ (0.56 g/L) and CuSO₄ (2 mM), sodium lignosulphonate (10 g/L), and wheat straw extract (50 ml) as inducers added on day-12 and day-21 of the fermentation yielded the highest laccase activity of 6,16,512 U/L in 28 days (Table 2) and further fermentations were accordingly carried out with SWX media.

At the end of fermentation, the fungal biomass was separated by filtration. The cell free broth containing secreted LMEs was used for the biomass pre-treatment studies.

The resultant optimized media was used for surface, static or submerged fermentation.

Preparation of Wheat/Rice straw extract: For adding as inducers, wheat/rice straw extracts were prepared by was soaking 150 g wheat/rice straw in distilled water (1500 ml) and incubating at 25° C. with continuous stirring at 150 rpm for a duration of 5 h. The mixture was filtered, and the resulting filtrate was centrifuged at 4500 rpm for 10 min at 25° C. The supernatant obtained was sterilized at 121° C. for 20 min. Extract was stored at −80° C. in aliquots (50 ml) until further use.

TABLE 2
Media compositions and media optimisation

| | SYM | FFD | SLS | SWX |
|---|---|---|---|---|
| | (Sucrose | (Full | (Sodium | (SLS |
| | Yeast | factorial | ligno- | with wheat |
| | extract | design | sulphonate | straw |
| Name | maltose) | medium) | medium) | extract*) |
| Medium | 10 g/L | Total sugars | Total sugars | Total sugars |
| composition | sucrose, | (5, or 20 g/L) | (56.2 g/L) from | (56.2 g/L) |
| | 4 g/L yeast | from cane | cane molasses, | from cane |
| | extract, | molasses, | yeast extract (1 | molasses, |
| | and 10 | corn steep | g/L), corn | yeast extract |
| | g/L malt | liquor (5.0 or | steep liquor (27 | (1 g/L), corn |
| | extract | 22 g/L), and | g/L), and | steep liquor |
| | | yeast extract | FeSO₄ (0.56 | (27 g/L) and |
| | | (1.0 g/L) | g/ L) | FeSO₄ (0.56 |
| | | | | g/ L) |
| Inducers | None | Tween 80 | CuSO₄ (1.2 | CuSO₄(1.2 |
| | | (w/v) (0.5%), | mM), sodium | mM), sodium |
| | | CuSO₄ (0.2 | lignosulphonate | lignosulphonate |
| | | mM) | (10 g/L) | (10 g/L) |
| | | | | and wheat |
| | | | | straw extract |
| | | | | (50 mL) |
| | | | | added on day |
| | | | | 12 and 21. |
| Maximum | 2,041 | 23,889 +/− | 1,45,665 U/L | 6,16,592 U/L |
| laccase | U/L in 20 | 5358 U/Lin | in 25 days | in 28 days |
| activity | days | 20 days | | |

### Example 5

### Compositional Analysis of Rice Straw

Analysis of carbohydrates and lignin content was done using Klason lignin method. The method uses two hydrolysis steps namely primary hydrolysis and a secondary hydrolysis. The analysis of composition was carried out using the shredded rice straw with particle size of 1-5 mm. The biomass was washed and air dried completely.

In the primary hydrolysis step, sample (3.0 mg) was mixed 3.0 ml of 72% H₂SO₄ in glass test tubes. The test tubes were incubated on a mechanical shaker at 100 rpm for one hour at about 32° C. In the secondary hydrolysis step, hydrolysate from first step was transferred to 100 ml glass bottles and diluted to 4% by adding H₂O (84 ml). The bottles were autoclaved at 121° C. for one hour. The autoclaved slurry was filtered and the filter papers with the residue were transferred to the pre-weighed crucibles in the muffle furnace at 575° C. overnight. Weight of the ash remaining in the crucibles was used to calculate acid insoluble lignin. The filtrate was used for HPLC analysis of glucose, xylose and arabinose.

### Example 6

### Pre-Treatment and Enzymatic Saccharification:

Step 1. Milling of Rice straw: Rice straw (Oryza sativa) collected from Mulshi area, Pune district, India was reduced to smaller size (10 to 30 mm) by shredding or grinding. Compositional analysis indicated presence of three major components: 1) Cellulose. 2. Hemicellulose, 3. Lignin as shown in Table 3.

TABLE 3
Compositional analysis of rice straw (% w/ w)

| S. No | Total solids | Quantity (g) |
|---|---|---|
| 1 | Cellulose | 35.0 |
| 2 | Xylan | 17.3 |
| 3 | Lignin | 14.2 |
| 4 | Arabinan | 2.7 |
| 5 | Ash | 9.4 |
| 6 | Silica | 5.5 |
| 7 | Protein | 3.3 |
| 8 | Extractive | 8.2 |
| 9 | Other | 4.5 |
| 10 | Total Solids | 94.60 |
| 11 | Moisture | 5.40 |

Step 2. Water washing: Biomass (900 g) as obtained above was mixed in about 24 L water (23999.71 g) (Total Solids (%)=3.61) for 20 min at 80° C. Solid liquid separation was carried out. The filtrate obtained was further used for the analysis of total solids (Table 4)

TABLE 4
Analysis of rice straw after water washing

| S. No | Total solids | Quantity (g) |
|---|---|---|
| 1 | Cellulose | 297.56 |
| 2 | Xylan | 147.29 |
| 3 | Lignin | 120.64 |
| 4 | Moisture | 23.30 |
| 5 | Arabinan | 19.76 |
| 6 | Ash | 75.74 |
| 7 | Silica | 44.71 |
| 8 | Protein | 27.05 |
| 9 | Extractive | 139.40 |
| 10 | Other Solids | 4.54 |

Step 3. Drying: Wet biomass (3547.4 g) obtained after solid-liquid separation was dried in an oven overnight at 65° C. and composition was analysed for total solids. 720 g of rice straw was obtained at this stage (Table 5).

TABLE 5
Analysis of rice straw for total solids after drying

| S. No. | Total solids | Quantity (g) |
|---|---|---|
| 1 | Cellulose | 256.16 |
| 2 | Xylan | 114.09 |
| 3 | Lignin | 91.31 |
| 4 | Moisture | 36.00 |
| 5 | Arabinan | 17.51 |
| 6 | Ash | 34.20 |
| 7 | Silica | 34.61 |
| 8 | Protein | 30.10 |
| 9 | Extractive | 102.60 |
| 10 | Other Solids | 3.42 |

Step 4. Pro-treatment using LME: The biomass from step 3 was distributed and added to three stainless steel reactors that are placed in water bath with temperature control. 280 g biomass was added to each of the three reactors and water was added final mass of 4 kg (is 7% Total Solids). After pre-treatment with LME and solid liquid separation, biomass was washed in alkaline peroxide solution (45 g NaOH and 23 g H₂O₂ in 2800 ml water) and incubated at 80° C. for 90 min. Table 6 depicts the compositional analysis of the samples after pre-treatment.

TABLE 6
Analysis of total sollds after enzymatic treatment

| S. | | LME treatment, | Water and ALP |
|---|---|---|---|
| No. | Composition | ALP treatment (g) | treatment (g) |
| 1 | Cellulose | 30.55 | 30.8 |
| 2 | Xylan | 10.37 | 9.5 |
| 3 | Lignin | 3.85 | 3.9 |
| 4 | Moisture | 216.49 | 266.8 |
| 5 | Arabinan | 1.35 | 1.3 |
| 6 | Ash | 0.25 | 0.4 |
| 7 | Silica | 0.40 | 0.5 |
| 8 | Protein | 0.30 | 0.3 |
| 9 | Extractive | 1.00 | 1.0 |
| 10 | Other Solids | 0.45 | 5.0 |

Step 5. Saccharification: Water treated biomass, ALP treated biomass and LME+ALP treated biomass obtained after solid liquid separation and water washing, were incubated with cellulase cocktail comprising cellulase, hemicellulase and xylanase for 72 h and further analysed for efficiency of conversion of cellulose to glucose and xylan to xylose.

### Example 7

Conversion efficiency of cellulose to glucose with or without water wash: Saccharification of laccase treated rice straw was compared with a in presence of a water wash and in absence of water wash. The washed sample yielded 9% higher cellulose to glucose conversion efficiency and 11% higher xylan to xylose conversion efficiency when compared to efficiency of conversion using un-washed rice straw (Table 7).

TABLE 7
Comparison of conversion efficiency of cellulose with and without washing

| | Glucose | Glucose | Xylose | Xylose |
|---|---|---|---|---|
| | efficiency (%) | efficiency (%) | efficiency (%) | efficiency (%) |
| | (No wash) | (Water wash) | (No wash) | (Water wash) |
| Test - Laccase | 21 ± 0.78 | 30 ± 7.29 | 6 ± 0.5 | 16.7 ± 9.6 |
| treated | | | | |
| Control - | 21 ± 2.54 | 20 ± 2.78 | 8 ± 1.2 | 8.7 ± 1.7 |
| Water treated | | | | |

### Example 8

Conversion efficiency of cellulose to glucose with and without peroxide treatment: Rice straw (7%) was incubated either in water with laccase treated (test) for 24 h at 45° C. and cellulase was added at 500 mg/g of cellulose. Glucose concentrations and % conversion of cellulose to glucose were measured after 3 days of saccharification for ‘no laccase control’ and ‘laccase treated’ biomass (Table 8)

TABLE 8
Cellulose to glucose conversion efficiency
after LME and ALP treatment

| | Test | |
|---|---|---|
| Conversion | (LME + ALP + | Control |
| efficiency (%) | Cellulase) | (ALP + Cellulase) |
| Cellulose to | 53 | 30 |
| glucose | | |

### Example 9

Determination of LME remaining after pre-treatment: Activity of laccase before and after one cycle of pre-treatment was measured. About 30% residual laccase activity was observed after pre-treatment (Table 9).

TABLE 9
Comparison of laccase activity before
and after one cycle of pre-treatment

| | Laccase (U/L) |
|---|---|
| Initial activity | 4200 |
| Activity after one use (recycled) | 1253 ± 81 |
| % residual activity | 30 ± 2 |

### Example 10

LME recycle: After the pre-treatment, LME was collected during solid liquid separation and the laccase activity was evaluated to estimate residual LME. This was used to determine course for a second round of pre-treatment. Both the pre-treated samples were subjected to saccharification and cellulose to glucose conversion efficiencies were estimated (Table 10).

TABLE 10
Evaluation of efficiency of recycled LME

| | % Efficiency of cellulose to glucose |
|---|---|
| | conversion |
| Water treated | 14.56 ± 1.44 |
| (Control) | |
| Water recycled | 3.89 ± 0 |
| (Control) | |
| LME treated | 23.54 ± 3.11 |
| Recycled LME | 32.68 ± 0.24 |

### Example 11

### Effect of Laccase

Scanning electron mirosopic images of rice straw treated with water (control) and Trametes versicolor laccase indicated distinct differences in physical changes on the surface as shown in FIG. 3.

### Example 12

Use of swollenin, expansin and AA9 in saccharification: Upon introducing the accessory proteins along with cellulases, LME pre-treated biomass yielded higher cellulose to glucose conversion efficiencies (Tables 11 and 12).

TABLE 11
Synergistic effect of accessory proteins on LME
pre-treated biomass during saccharification

| | % Efficiency of cellulose to glucose |
|---|---|
| Treatment | conversion |
| Water treated (control) | 31.52 |
| LME treated | 26.86 |
| ALP treated | 36.56 |
| LME + ALP | 47.00 |
| LME + ALP + Swo TP | 76.85 |
| LME + ALP + Swo TH | 78.71 |
| LME + ALP + Swo TR | 78.9 |
| LME + ALP + Swo TR (FERM) | 69.57 |

TABLE 12
Use of purified expansin and AA9 saccharification

| | % Cellulose to glucose conversion |
|---|---|
| Treatment | efficiency. |
| Water treated (control) | 35.63 |
| ALP treated | 46.82 |
| Bs-Expansin | 39.92 |
| Bs-Exp + ALP | 80.39 |
| LME + Bs-Exp + ALP | 83.56 |
| AA9 (control) | 30.78 |
| AA9 + ALP | 71.62 |
| LME + AA9 + ALP | 86.55 |

We claim:

1. A process for releasing carbohydrates/sugars from agricultural feedstock, the process comprising:
processing the agricultural feedstock;
pre-treating the processed feedstock with lignin modifying enzymes (LME) to produce a reaction mixture;
separating the reaction mixture into solids and liquid comprising LME;
washing the solids with at least one of water, alkaline peroxide or ionic liquid to obtain washed solids free from LME; and
saccharifying the washed solids by hydrolysis with one or more cellulolytic enzymes to release carbohydrates/sugars at an increased efficiency.

2. The process as claimed in claim 1, wherein the lignin modifying enzymes (LME) are produced by fungi and include one or more of lignin peroxidase (Lip), manganese peroxidase (MnP), versatile peroxidases (VP) and laccases.

3. The process as claimed in claim 1, wherein the lignin modifying enzymes (LME) are produced by fermentation of white rot fungi, wherein the strains of white rot fungi are selected from Trametes versicolor, Pleurotus ostreatus, Phanerochaete chrysosporium, and Penicillium chrysogenum.

4. The process as claimed in claim 3, wherein the lignin modifying enzymes (LME) are produced using surface, static or submerged fermentation.

5. The process as claimed in claim 3, wherein the lignin modifying enzymes (LME) are produced using submerged fermentation.

6. The process as claimed in claim 5, wherein the submerged fermentation media comprises molasses, yeast extract, corn steep liquor, sodium lignosulphonate and FeSO₄.

7. The process as claimed in claim 5, wherein the submerged fermentation is carried out from 7 to 25 days at a pH ranging from 5.5 to 6.5 and at a temperature ranging from 18 to 37° C. by inoculating the fermentation media with 5 to 10% active mycelia to provide LME.

8. The process as claimed in claim 5, wherein the submerged fermentation media optionally further comprises one or more inducers added at suitable times during the fermentation.

9. The process as claimed in claim 8, wherein the inducers are selected from copper sulphate, wheat straw extract, rice straw extract, lignosulphonate, and 2,5-xylidine.

10. The process as claimed in claim 5, wherein the mycelia and other solids are separated using solid liquid separation to provide clear broth containing LME.

11. The process as claimed in claim 1, wherein the agricultural feedstock is selected from one or more of rice straw, corncob, corn stover, sugarcane/beet bagasse, wheat straw or combinations thereof.

12. The process as claimed in claim 1, wherein the processing of agricultural feedstock involves one or more steps of treating with hot water or mild acid drying and mechanical shredding to provide solid particles having a size of <40 mm.

13. The process as claimed in claim 1, wherein the pre-treating of the processed feedstock includes mixing the processed feedstock with the LME from 10 hours to 48 hours at a temperature ranging from 40° C. to 50° C. to produce the reaction mixture.

14. The process as claimed in claim 1, wherein separating the reaction mixture includes filtration, centrifugation, decantation methods to provide solids and liquid comprising LME.

15. The process as claimed in claim 1, wherein treating the solids comprises one or more washes to provide the solids free from LME.

16. The process as claimed in claim 1, wherein washing of the solids with alkaline peroxide comprises 0.2% to 1% w/w NaOH and 0.1% to 2% w/w H₂O₂ based on/with respect to cellulose content at a temperature ranging from 60° C. to 80° C. for 60 to 90 minutes to obtain washed solids.

17. The process as claimed in claim 1, wherein saccharifying the washed solids involves hydrolysis with one or more cellulolytic enzymes selected from cellulase, hemicellulase or xylanase.

18. The process as claimed in claim 17, wherein hydrolysis includes mixing washed solids with one or more of cellulase, hemicellulase or xylanase to obtain a slurry having 5% to 15% w/w solids at pH of 4.5 to 5.5 and temperature of 40° C. to 80° C. for 24 to 120 hours to obtain a final stream comprising carbohydrates/free sugars.

19. The process claimed in 17, wherein hydrolysis involves addition of one or more accessory proteins selected from expansins, swollenins and lytic polysaccharide monosaccharides (LMPO) such as AA9.

20. The process as claimed in claim 17, wherein the hydrolysis efficiency is in the range of about 40% to 80%.

21. The process as claimed in claim 1, wherein the free carbohydrates/sugars are further fermented using yeast for 24 to 72 hrs at 30° C. with continuous agitation to obtain ethanol.

22. The process as claimed in claim 1, wherein the carbohydrates/free sugars are used for producing other biochemicals including furfural, HMF, lactic acid or 2,3 BDO.

23. The process as claimed in claim 1, wherein the separated liquid comprising LME comprises about 40 to 60% laccase that is recycled back for next cycle of biomass pre-treatment.
